(19) United States
(12) Patent Application Publication
(10) Pub. No.: US 2026/0227331 A1
(43) Pub. Date: Aug. 6, 2026

(54) POROUS METAL COMPLEX-CONTAINING FILM, DETECTION ELEMENT PROVIDED WITH SAME, AND METHOD OF PRODUCING POROUS METAL COMPLEX-CONTAINING FILM

(71) Applicant: TAIYO NIPPON SANSO CORPORATION, Shinagawa-ku, Tokyo (JP)

(72) Inventors: Susumu TOKAIRIN, Shinagawa-ku, Tokyo (JP); Tatsumi IWAMOTO, Shinagawa-ku, Tokyo (JP)

(73) Assignee: TAIYO NIPPON SANSO CORPORATION, Shinagawa-ku, Tokyo (JP)

(21) Appl. No.: 19/149,846

(22) PCT Filed: Dec. 12, 2023
(86) PCT No.: PCT/JP2023/044468

§ 371 Date: Jul. 22, 2025

(30) Foreign Application Priority Data
Mar. 6, 2023 (JP) 2023-034089

(51) Int. Cl. G01N 21/59; G01N 33/00

(52) U.S. Cl. CPC G01N 21/59; G01N 33/0036

(57) ABSTRACT

A porous metal complex-containing film that can be produced through a simple process, has a high yield during production, and exhibits high detection sensitivity when used as a detection element, along with a detection element provided with the same and a method of producing the porous metal complex-containing film, are provided. The porous metal complex-containing film includes a filter having a void inside and having light permeability and gas permeability, and a porous metal complex fixed to the void. The porous metal complex contains a metal ion and an organic ligand that coordinates to the metal ion. A median diameter of the porous metal complex on a volume basis is 0.1 μm or more and 3 μm or less.

### TECHNICAL FIELD

The present disclosure relates to a porous metal complex-containing film, a detection element provided with the same, and a method of producing a porous metal complex-containing film.

### BACKGROUND

A porous metal complex (also referred to as porous organic metal framework material, metal-organic framework, etc.; hereinafter also referred to as MOF) is a polymeric metal complex obtained by crosslinking metal ions with organic ligands.

JP 2017-512891 A (Patent Literature (PTL) 1) discloses a porous film containing a porous organic metal framework material (MOF), a composition for preparing this film, and a method of using the same. This porous film contains a material that is at least one type of porous organic metal framework material in an amount of 51 mass % to 99.9 mass % based on the total mass of the film, the material including at least one type of an at least bidentate organic compound coordinated to at least one metal ion. The porous film also contains at least one type of fibrillated fluoropolymer in an amount of 0.1 mass % to 49 mass % based on the total mass of the film, and an additive component in an amount of 0 mass % to 48.9 mass % based on the total mass of the film. The uses of this film include applications as sensors, conductive films, and storage or separation devices. This film is produced by mixing commercially available porous metal complexes with fluoropolymer powder to obtain a powder mixture, then treating the powder mixture in a pestle to fibrillate the fluoropolymer contained in the powder mixture, and finally calendaring the resulting paste-like mass to produce a thin film.

JP 2019-163228 A (PTL 2) discloses a metal-organic framework (MOF), a phosphor film, and a molecular detection device. This MOF is a metal-organic framework that emits fluorescence and deforms due to interaction with a target molecule, has a pillared layer structure, and includes metal ions, tetradentate ligands bonded to the metal ions, and bidentate ligands bonded to the metal ions. The pillared layer structure has a plurality of two-dimensional layered structures formed by metal ions and first ligands having carboxyl groups, which are crosslinked by second ligands having pyridyl, imidazole, or amino groups, resulting in a three-dimensional structure. This MOF is excited by light from a light source to emit fluorescence and deforms due to interaction with guest molecules, which are the target molecules. The emission spectrum of fluorescence from the MOF changes according to the aforementioned deformation. PTL 2 discloses that molecular detection devices such as VOC sensors and explosive sensors can be constructed by utilizing the above phenomenon. Moreover, it is said that the pillared layer structure enables the detection of target molecules with high sensitivity.

JP 2020-32325 A (PTL 3) discloses a selective gas permeable film having a metal-organic framework (MOF) layer and a method of producing the same. This selective gas permeable film has gas permeable films closely arranged on both sides of a layer of metal-organic framework (MOF) particles with gas selectivity. In PTL 3, Cu(bim)₂ is exemplified as an MOF that can selectively separate and recover CO₂ from a CH₄/CO₂ mixed gas. As a method of synthesizing Cu(bim)₂ particles, PTL 3 discloses the case in which 30 mg of terephthalic acid and 30 mg of copper(II) nitrate trihydrate are added while stirring into a mixed solution of 4 ml of dimethylformamide and 4 ml of acetonitrile, then placed in a constant temperature bath at 40° C. for 24 hours, followed by centrifugation to obtain 50 mg of Cu(bim)₂ particles with an average particle diameter of about 100 nm.

### CITATION LIST

### Patent Literature

PTL 1: JP 2017-512891 A
PTL 2: JP 2019-163228 A
PTL 3: JP 2020-32325 A

### SUMMARY

### Technical Problem

In the porous film disclosed in PTL 1, the porous metal complex may be destroyed due to shear stress during the fibrillation process, which can lead to broadening of the particle size distribution and a decrease in crystallinity. Additionally, when the porous metal complex is beaten in the fibrillation process, fine powder of the porous metal complex may enter the voids of the fluoropolymer and the porous metal complex, causing the voids to become blocked. As a result, high sensitivity detection may not be achievable when using the porous film as a sensor.

The molecular detection device disclosed in PTL 2 has a pillared layer structure with a plurality of two-dimensional layered structures and uses a plurality of MOFs, making the molecular detection process complex. Therefore, stable high sensitivity detection may not be achievable.

When forming an MOF particle layer like the selective gas permeable film disclosed in PTL 3, a method exists for obtaining MOF particles by centrifugation after synthesis, but as in the synthesis method of Cu(bim)₂ particles (MOF particles) disclosed in PTL 3, processes such as crystal growth and centrifugation are required, complicating the entire process and potentially reducing yield.

In light of the above-described conventional technology, it is desirable to provide a porous metal complex-containing film that can be produced through a simple process, has a high yield during production, and is highly sensitive, and a method of producing the same. Furthermore, it is desirable to provide a detection element using such a porous metal complex-containing film.

The present disclosure has been made in view of such circumstances, and it is an aim thereof to provide a porous metal complex-containing film that can be produced through a simple process, has a high yield during production, and exhibits high detection sensitivity when used as a detection element, along with a detection element provided with the same and a method of producing the porous metal complex-containing film.

### Solution to Problem

The porous metal complex-containing film according to the present disclosure for achieving the above aim includes
a filter having a void inside and having light permeability and gas permeability; and
a porous metal complex fixed to the void, wherein
the porous metal complex contains a metal ion and an organic ligand that coordinates to the metal ion, and
a median diameter of the porous metal complex on a volume basis is 0.1 μm or more and 3 μm or less.

A detection element according to the present disclosure for achieving the above aim includes
the aforementioned porous metal complex-containing film.

A method of producing a porous metal complex-containing film according to the present disclosure for achieving the above aim is
a method of producing the aforementioned porous metal complex-containing film and includes:
a solution preparation process of mixing a first polar solvent, the organic ligand, and a metal salt to prepare a precursor solution of the porous metal complex;
an impregnation process of impregnating the precursor solution into the filter to obtain a precursor-impregnated film;
an intermediate drying process of drying the precursor-impregnated film to obtain a precipitated film; and
a washing process of washing the precipitated film with a second polar solvent to obtain a washed film.

### Advantageous Effect

According to the present disclosure, a porous metal complex-containing film that can be produced through a simple process, has a high yield during production, and exhibits high detection sensitivity when used as a detection element, along with a detection element provided with the same and a method of producing the porous metal complex-containing film, can be provided.

### BRIEF DESCRIPTION OF THE DRAWINGS

In the accompanying drawings:

FIG. 1 is an explanatory diagram of a trace moisture concentration measurement apparatus using the porous metal complex-containing film according to the present embodiment as a detection element; and

FIG. 2 is a bar graph illustrating the product yield of Examples and a Comparative Example.

### DETAILED DESCRIPTION

With reference to the drawings, a porous metal complex-containing film according to the present embodiment, a detection element provided with the same, and a method of producing the porous metal complex-containing film are described.

First, an overview of the porous metal complex-containing film according to the present embodiment, the detection element provided with the same, and the method of producing the porous metal complex-containing film will be provided.

The porous metal complex-containing film according to the present embodiment includes a filter having a void inside and having light permeability and gas permeability, and a porous metal complex fixed to the void. The porous metal complex contains a metal ion and an organic ligand that coordinates to the metal ion. The median diameter of the porous metal complex on a volume basis is 0.1 μm or more and 3 μm or less.

A detection element according to the present embodiment includes the porous metal complex-containing film according to the present embodiment.

This porous metal complex-containing film can be produced by the following production method as an example. Specifically, the method of producing the porous metal complex-containing film according to the present embodiment includes a solution preparation process of mixing a first polar solvent, the organic ligand, and a metal salt to prepare a precursor solution of the porous metal complex, an impregnation process of impregnating the precursor solution into the filter to obtain a precursor-impregnated film, an intermediate drying process of drying the precursor-impregnated film to obtain a precipitated film, and a washing process of washing the precipitated film with a second polar solvent to obtain a washed film.

The porous metal complex-containing film according to the present embodiment can be produced through a simple process, has a high yield during production, and exhibits high detection sensitivity when used as a detection element (sensor).

Hereinafter, the porous metal complex-containing film according to the present embodiment, the detection element provided with the same, and the method of producing the porous metal complex-containing film will be described in detail.

The porous metal complex-containing film according to the present embodiment includes a filter having voids inside and a porous metal complex (MOF) fixed in the voids. The porous metal complex-containing film according to the present embodiment can, for example, be suitably used as a detection element for detecting water contained in gas and measuring the concentration of the water.

The filter is a substrate for forming the porous metal complex-containing film according to the present embodiment.

The filter has light permeability and gas permeability. The material of the filter is not particularly limited. Examples of the material of the filter include metal, ceramic, glass, wood, resin, paper, and fabric. The material of the filter is preferably cellulose fiber, glass fiber, or polytetrafluoroethylene fiber. In particular, polytetrafluoroethylene fiber is suitable as the material of the filter.

The filter may be formed in a plate-like or film-like shape. The thickness of the filter is not particularly limited, but the filter may be formed in a thin plate-like shape. The thickness of the filter may be, for example, 100 μm or more and 5 mm or less.

As described above, the filter has voids inside. The filter may, for example, be porous. The porosity of the filter may be determined considering the physical properties, such as flexibility and mechanical strength, of the porous metal complex-containing film required when the filter is used as a substrate for the porous metal complex-containing film. The porosity of the filter may be 50% or more and 95% or less by volume.

The voids of the filter are fixed with fine particles of the porous metal complex described later. In the present embodiment, fixation refers to a state in which fine particles of the porous metal complex are captured in the voids of the filter, and the fine particles of the porous metal complex do not detach from the filter (porous metal complex-containing film) when the porous metal complex-containing film is used as a detection element. The manner in which the porous metal complex particles are fixed in the pores of the filter includes a case in which the porous metal complex particles do not detach from the pores due to steric hindrance within the pores, and a case in which the porous metal complex particles are supported on a surface without pores by adhesion, bonding, or spreading. It suffices for the voids in the filter to have a size such that spherical particles with a diameter of 10 μm or more cannot pass through (are captured).

The porous metal complex is a polymeric metal complex obtained by crosslinking metal ions with organic ligands. The porous metal complex is a porous material that has a plurality of pores communicating with the outside. In the present embodiment, the porous metal complex may be a structure in which metal ions or metal atoms are continuously bonded to organic ligands having two or more coordinating functional groups. Functional molecules or additives may be contained within the pores of the porous metal complex. In the case of encapsulating functional molecules or additives in the pores of a porous metal complex, it should be ensured that the voids of the pores do not become blocked from communicating with the outside.

Examples of the metal ions or metal atoms forming part of the porous metal complex include transition metals, aluminum, and magnesium. The metal ions or metal atoms forming part of the porous metal complex are not limited to one type. Two or more types of metal ions or metal atoms may form part of the porous metal complex.

The metal ions forming part of the porous metal complex are preferably copper ions.

The functional group of the organic ligands must be capable of coordinating to the aforementioned metal ion or metal atom. Examples of the functional group of an organic ligand capable of coordinating to the aforementioned metal ion or metal atom include a hydroxy group, an imidazole group, a sulfonic acid group, an amino group, a carboxyl group, and an amide group.

The organic ligand is particularly preferably benzene-1,3,5-tricarboxylic acid (in a coordinated state, 1,3,5-benzenetricarboxylate).

In the porous metal complex-containing film according to the present embodiment, the content of the porous metal complex is preferably 0.5% or more and 50% or less by mass, more preferably 1% or more and 30% or less, and even more preferably 1% or more and 15% or less.

The particle size of the porous metal complex, when viewed in terms of volume, is such that the median diameter (cumulative 50% particle size, i.e., d50) is 0.1 μm or more and 3 μm or less. The median diameter of the porous metal complex is preferably 0.1 μm or more and 2 μm or less. The particle size of the porous metal complex may be appropriately adjusted considering the securing of response intensity at the time of detection or sensing when using the porous metal complex-containing film as a detection element, and the maintenance of the crystallinity of the porous metal complex particles.

The porous metal complex-containing film according to the present embodiment can, for example, be produced by a production method that includes a solution preparation process, an impregnation process, an intermediate drying process, and a washing process, as described above. In this production method, the solution preparation process, impregnation process, intermediate drying process, and washing process are executed in this order. In this production method, after the washing process, the drying process described below may be performed. The intermediate drying process may include a humidification process described below.

As described above, the solution preparation process is a process of mixing a polar solvent (which is an example of the first polar solvent and is hereinafter referred to as the first polar solvent), an organic ligand, and a metal salt to prepare a precursor solution of the porous metal complex.

The precursor solution is prepared by dissolving the organic ligand and the metal salt in the polar solvent. At the time of dissolving, the order in which the organic ligand and the metal salt are added to the polar solvent, the order in which they are dissolved, and the order in which they are mixed are not limited. For example, the metal salt may be dissolved after dissolving the organic ligand in the polar solvent. Alternatively, the organic ligand may be dissolved after dissolving the metal salt in the polar solvent. Additionally, a porous metal complex precursor solution may be obtained by mixing a solution of an organic ligand dissolved in a polar solvent with a solution of a metal salt dissolved in a polar solvent.

The first polar solvent is not limited as long as it can dissolve the organic ligand and the metal salt. Examples of the first polar solvent include tetrahydrofuran, acetonitrile, N,N-dimethylformamide, acetone, methanol, ethanol, 1-propanol, 2-propanol, 1-butanol, and 2-butanol. The first polar solvent is preferably an organic solvent.

In particular, in the case of using copper nitrate as the metal salt and 1,3,5-carboxylic acid as the organic ligand, methanol, ethanol, 1-propanol, 2-propanol, 1-butanol, and 2-butanol are preferable. To obtain crystals of porous metal complexes with a particularly small particle size distribution (little variation in particle size), methanol, ethanol, 1-propanol, 2-propanol, 1-butanol, or 2-butanol is preferable.

The impregnation process is a process of impregnating a filter with the precursor solution to obtain a precursor-impregnated film as described above.

The precursor-impregnated film is produced by impregnating the filter with the precursor solution. The method of impregnating the filter with the precursor solution is not particularly limited. As an example, the precursor solution may be filled in a container, and the filter may be immersed in the precursor solution in the container to impregnate the filter with the precursor solution. Alternatively, the precursor solution may be dripped onto the filter to impregnate the filter with the precursor solution. During impregnation, the atmosphere may be reduced in pressure or heated. The atmosphere may also be heated while being reduced in pressure during impregnation.

If the precursor solution does not easily seep into the voids of the filter, the filter may be impregnated in advance with a polar solvent other than the precursor solution (for example, one selected from the first polar solvents mentioned above, hereinafter also referred to as the third polar solvent), and the precursor solution may then be impregnated into the filter.

The intermediate drying process is a process of drying the precursor-impregnated film to precipitate the porous metal complex. There is no particular limitation on the method of drying the precursor-impregnated film in the intermediate drying process. For example, the precursor-impregnated film may be dried under reduced pressure in a vacuum dryer. The precursor-impregnated film may also be dried while being heated. In the case of heating during the intermediate drying process, the precursor-impregnated film is preferably heated to a temperature of 40° C. or higher and 100° C. or lower. In the intermediate drying process, the porous metal complex precipitates in the voids of the filter. Hereinafter, the precursor-impregnated film after the intermediate drying process is also referred to as the precipitated film.

The intermediate drying process may include a humidification process that exposes the dried precursor-impregnated film to a water vapor-containing gas to humidify the dried precursor-impregnated film. Hereinafter, the precursor-impregnated film that has been dried at least once is also referred to as the pre-precipitated film. Furthermore, hereinafter, exposing the precursor-impregnated film to a water vapor-containing gas to humidify the precursor-impregnated film may simply be described as performing a humidification treatment. The precursor-impregnated film after the humidification treatment is also referred to as the humidified film. In some cases, the humidification process narrows (sharpens) the particle size distribution of the porous metal complex fixed in the voids of the filter.

If humidification treatment is performed during the intermediate drying process, it is preferable to dry the humidified film again to obtain the precipitated film (the precipitated film as the humidified film).

If no humidification treatment is performed during the intermediate drying process, the pre-precipitated film may become the precipitated film. If no humidification treatment is performed during the intermediate drying process, the pre-precipitated film may be additionally dried to become the precipitated film.

That is, the intermediate drying process is a process of at least drying the precursor-impregnated film to obtain a precipitated film in which the porous metal complex is precipitated in the voids of the filter. The intermediate drying process preferably includes a first intermediate drying process of obtaining the pre-precipitated film, a humidification process of humidifying the pre-precipitated film to obtain the humidified film, and a second intermediate drying process of drying the humidified film to obtain the precipitated film. The intermediate drying process may omit the humidification process and include a first intermediate drying process of obtaining the pre-precipitated film and a second intermediate drying process of further drying the pre-precipitated film to obtain the precipitated film.

The second intermediate drying process can suppress variations in particle size of the porous metal complex in the subsequent washing process. The second intermediate drying process may also make the subsequent washing process more efficient.

Details of the first intermediate drying process, the second intermediate drying process, and the humidification process in the intermediate drying process are now provided.

The method of drying the precursor-impregnated film in the first intermediate drying process is not particularly limited, but for example, the precursor-impregnated film may be dried under reduced pressure in a vacuum dryer. The first intermediate drying process may be performed while heating the precursor-impregnated film under a flow of carrier gas. The first intermediate drying process may be performed under reduced pressure while heating the precursor-impregnated film under a flow of carrier gas. In the first heating process, the precursor-impregnated film is preferably heated to a temperature of 60° C. or higher and 80° C. or lower.

The method of additional drying of the pre-precipitated film or the method of drying the humidified film in the second intermediate drying process is not particularly limited, but for example, the precursor-impregnated film may be dried under reduced pressure in a vacuum dryer. The second intermediate drying process may be performed while heating the precursor-impregnated film under a flow of carrier gas. The second intermediate drying process may be performed under reduced pressure while heating the precursor-impregnated film under a flow of carrier gas. In the second intermediate drying process, the pre-precipitated film or the humidified film is preferably heated to a temperature between 40° C. and 100° C.

The drying of the precursor-impregnated film, pre-precipitated film, or humidified film in the first intermediate drying process and the second intermediate drying process within the intermediate drying process may, for example, involve a gas supplier such as a gas cylinder that supplies a carrier gas; a processing container, such as a round-bottom flask, having a processing chamber that contains the precursor-impregnated film, pre-precipitated film, or humidified film and into which the carrier gas is supplied from the gas supplier; a heating unit having a heating mechanism such as a heater or oil bath for heating these films contained in the processing chamber of the processing container; and a pump that reduces the pressure of the internal atmosphere in the processing chamber of the processing container.

Examples of a suitable carrier gas in the first intermediate drying process and second intermediate drying process are hydrogen, nitrogen, argon, oxygen, helium, carbon dioxide, hydrocarbons, or clean dry air (air).

The humidification treatment in the humidification process may be performed by exposing the pre-precipitated film to a water vapor-containing gas adjusted to a predetermined temperature and humidity. Specifically, for example, the pre-precipitated film may be contained in a container (hereinafter also referred to as a humidification container) with an internal atmosphere adjusted to a predetermined temperature and humidity, thereby exposing the film after cleaning to the water vapor-containing gas. Additionally, while the pre-precipitated film is contained in the humidification container, a water vapor-containing gas adjusted to a predetermined temperature and humidity may be passed through the humidification container.

The humidification treatment may be performed using a thermo-hygrostat provided with a mechanism for circulating gas within a chamber and a device for controlling temperature and humidity of the gas. Alternatively, a supply pipe for temperature-and humidity-controlled gas and an exhaust pipe for discharging the gas may be connected to a container capable of housing the washed film, and the container may be used as a humidification container. The supply pipe may be equipped with a gas flow control device, a temperature regulator (such as an electric heater or cooling device), and a humidity regulator (such as a water vapor generator like a bubbler or a gas bypass pipe). The exhaust pipe may be provided with a drain trap as needed. The temperature of the humidification container may be adjusted by, for example, a water bath or an oil bath.

The gas supplied to the humidification container before humidification (the carrier gas for transporting the water vapor) is not particularly limited. Examples of the gas supplied to the humidification container include hydrogen, nitrogen, argon, oxygen, helium, carbon dioxide, and clean dry air (air). Use of inert gases, such as nitrogen and argon, is particularly preferable.

It suffices for the flow control device to be capable of controlling the gas flow rate. Examples of the flow control device include a mass flow controller, a needle valve, and a diaphragm valve. If more precise control is required, it is preferable to use a mass flow controller.

The water vapor generator is not particularly limited as long as it can generate any amount of water vapor. Examples of the water vapor generator include the aforementioned bubbler or boiler. A bubbler is a device or mechanism that generates gas containing water vapor by releasing gas (bubbling) into water stored in a container. To accurately control the amount of water vapor contained in the gas, it is preferable for the bubbler to be able to freely control the temperature of the gas being bubbled or the water stored in the container.

The atmosphere in which the pre-precipitated film is exposed to water vapor, that is, the temperature of the water vapor-containing gas is preferably 30° C. or higher and 70° C. or lower. Also, the humidity of the water vapor-containing gas is preferably 20% or higher and 70% or lower in terms of relative humidity. In the following description, the explanation of humidity is based on relative humidity. The temperature and humidity of the water vapor-containing gas are more preferably 40° C. or higher and 60° C. or lower, and 30% or higher and 70% or lower, and even more preferably 50° C. or higher and 60° C. or lower, and 40% or higher and 60% or lower.

The time for exposing the pre-precipitated film to the water vapor-containing gas is preferably between 12 hours and 72 hours, and more preferably between 12 hours and 24 hours.

The washing process is a process of washing the precipitated film with a polar solvent (which is an example of the second polar solvent and is hereinafter referred to as the second polar solvent) to obtain the washed film.

The washing of the precipitated film is performed with the second polar solvent. By washing the precipitated film, it is possible to remove microparticles of the porous metal complex that are not immobilized in the voids of the filter, and raw material of the porous metal complex (metal ions and organic ligands) that is not crystallized.

The second polar solvent may be selected from the aforementioned examples of the first polar solvent. The second polar solvent may be the same as or different from the solvent used as the first polar solvent. The second polar solvent is preferably ethanol, methanol, or acetone.

The precipitated film may be washed by immersing the precipitated film in the second polar solvent or by dropping the second polar solvent onto the precipitated film to pass the second polar solvent through the precipitated film. During the washing of the precipitated film, the second polar solvent may be warmed or heated. For example, the precipitated film may be washed while the second polar solvent is heated and refluxed.

The particle size distribution of the porous metal complex fixed in the voids of the filter may be determined by image analysis using a scanning electron microscope (SEM).

The particle size distribution and median diameter of the porous metal complex in the present embodiment were obtained by measuring the maximum diameter of the crystals of the porous metal complex present in an image captured using a scanning electron microscope and calculating the volume-based particle size distribution based on this maximum diameter.

In the present embodiment, the production yield of the porous metal complex-containing film may be determined based on whether a predetermined detection sensitivity can be obtained when the porous metal complex-containing film is used as a detection element. That is, in the present embodiment, the yield of the porous metal complex-containing film production is the ratio of the number of porous metal complex-containing films that achieved the predetermined detection sensitivity to the total number of porous metal complex-containing films produced in a batch or lot.

An example of a device that can use the porous metal complex-containing film according to the present embodiment as a detection element is a device for measuring trace moisture concentration in a gas.

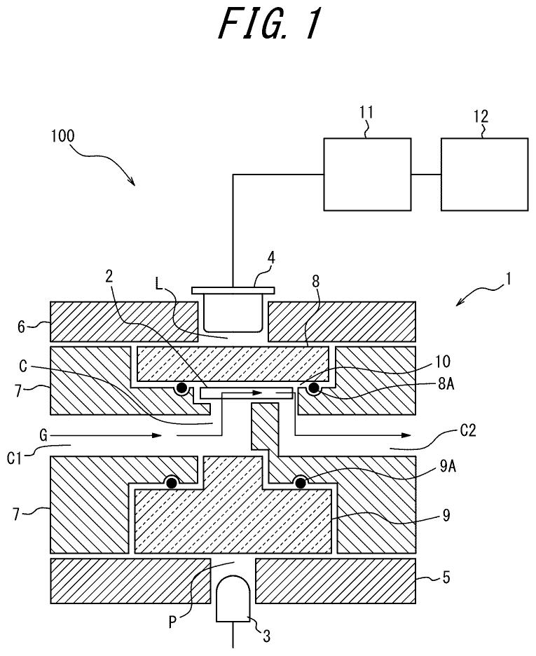

FIG. 1 illustrates an example of a trace moisture concentration measurement apparatus 100 that can use a porous metal complex-containing film according to the present embodiment as a detection element. In FIG. 1, for the purpose of illustrating the internal structure, a schematic cross-sectional configuration of a measurement unit 1 of the measurement apparatus 100 is illustrated.

The measurement unit 1 is configured by three metal blocks: a central block 7 that forms a sample gas path C through which the sample gas G to be measured flows, a light source side block 5 arranged on one side of the sample gas path C, and a light receiving side block 6 arranged on the other side of the sample gas path C.

The central block 7 is provided with a sample gas inflow path C1 for introducing the sample gas G, a sample gas outflow path C2 for guiding the sample gas G out, and a detection element holder 10 for holding the detection element 2, which is a porous metal complex-containing film.

The light source side block 5 has a measurement light path P with a light source 3 provided at the opening end. The light receiving side block 6 has a light receiving path L with a light receiving element 4 serving as a light receiver provided at the opening end. The light receiving path L is arranged on the axis of the measurement light path P. A convex light-transmitting member 9 is sandwiched, together with an annular packing 9A, between the light source side block 5 and the central block 7.

A flat light-transmitting member 8 is sandwiched, together with an annular packing 8A, between the light receiving side block 6 and the central block 7. The convex light-transmitting member 9 and the flat light-transmitting member 8 have light permeability and gas impermeability, and by placing the annular packings 8A and 9A between these members and the central block 7, airtightness is obtained to prevent leakage of the sample gas G from the sample gas path C to the outside and intrusion of external gas into the sample gas path C.

A light source that can emit measurement light with a wavelength of 200 nm or more and 800 nm or less, for example, can be used as the light source 3. The light source 3 may be an LED light source or a laser light source.

The light receiving element 4 measures the intensity of the measurement light that has passed through the detection element 2 from the sample gas path C to the light receiving path L, converts the intensity into a voltage signal, and outputs the result. Any light receiving element capable of measuring the intensity of the measurement light can be used as the light receiving element 4. Examples of the light receiving element 4 include a photodiode and a photomultiplier tube.

A voltage measurement unit 11 is connected to the light receiving element 4 to measure the voltage signal outputted from the light receiving element 4. A calculator 12 for performing various arithmetic processes based on the measured voltage signal is connected to the voltage measurement unit 11. The calculator 12 can calculate the amount of change over time in the optical properties of the detection element 2 based on the change in intensity of the measurement light that has passed through the detection element 2 to the light receiving path L, and can calculate the concentration of gaseous impurity components (for example, the moisture concentration) accurately using this amount of change over time.

When detecting the concentration of gaseous impurity components in the sample gas G using the measurement apparatus 100, the sample gas inflow path C1 is connected to the upstream side of the sample gas path C through which the sample gas G flows, and the sample gas outflow path C2 is connected to the downstream side of the sample gas path C. As a result, the sample gas G that has flowed from the upstream gas path into the sample gas inflow path C1 is introduced into the sample gas path C, and the sample gas G that has passed through the detection element 2 is guided out from the sample gas outflow path C2 to the downstream gas path, so that the sample gas G continuously flows within the sample gas path C.

While the sample gas G is flowing, the light source 3 is activated, and measurement light with a preset intensity is irradiated into the sample gas path C through the convex light-transmitting member 9 from the measurement light path P. The measurement light that has passed through the detection element 2 and the flat light-transmitting member 8 is received by the light receiving element 4, converted into a measurement voltage corresponding to the intensity of the received measurement light, and outputted to the voltage measurement unit 11. Voltage information is outputted from the voltage measurement unit 11 to the calculator 12. The calculator 12 outputs the concentration of a gaseous impurity component (detection result) based on this voltage information to a display device, such as a monitor or printer (not illustrated). The calculator 12 can calculate the accurate concentration of a gaseous impurity components using a relational expression, created in advance, between the intensity (voltage) of the measurement light at the light-receiving element 4 and the concentration of the gaseous impurity component (for example, the moisture concentration).

### EXAMPLES

A porous metal complex-containing film according to the present embodiment, a detection element provided with the same, and a method of producing the porous metal complex-containing film are described below based on Examples.

### Example 1

First, a precursor solution was prepared as follows.

First, ethanol (grade: special reagent grade) produced by Kanto Chemical Co., Inc. was weighed as a first polar solvent into a 25 mL container (made of perfluoroalkoxyalkane), and trimesic acid (benzene-1,3,5-tricarboxylic acid) produced by Kanto Chemical Co., Inc. was added and dissolved by stirring. Next, copper nitrate trihydrate manufactured by Kanto Chemical Co., Inc. was further added and dissolved by stirring to prepare a precursor solution. Copper nitrate trihydrate was added in an amount such that the molar ratio of copper nitrate trihydrate to benzene-1,3,5-tricarboxylic acid was 1.81. The atmosphere (environment) during mixing was set to a temperature of 20° C. and a relative humidity of 60%.

Next, a filter was cut out into a predetermined shape, and the precursor solution was impregnated into the cut-out filter. First, a circular polytetrafluoroethylene filter having a thickness of 1 mm and a diameter of 70 mm (PF100 produced by ADVANTEC) was punched out with a punch having a diameter of 7 mm to obtain a circular filter having a diameter of 7 mm. Then, the same ethanol as the first polar solvent was impregnated as a third polar solvent into the cut-out filter, and the precursor solution was subsequently impregnated to obtain a precursor-impregnated film.

Next, the precursor-impregnated film was placed in a petri dish made of perfluoroalkoxyalkane and dried by heating at 80° C. for 2 hours (first intermediate drying process). Subsequently, the precursor-impregnated film (pre-precipitated film) was transferred to a 100 mL glass round-bottom flask. The mouth of this round-bottom flask was then closed with a silicone stopper connected to a vacuum tube, and the vacuum tube was connected to a rotary oil pump. Subsequently, the round-bottom flask was heated to 100° C. in an oil bath while being evacuated with the vacuum pump to create a vacuum inside the round-bottom flask, and the contents were dried by vacuum-heating for 2 hours (second intermediate drying process) to obtain a precipitated film.

Next, the precipitated film was removed from the round-bottom flask used for vacuum heating and was transferred to another 100 mL round-bottom flask. Then, 20 mL of ethanol produced by Kanto Chemical Co., Inc. was added as a second polar solvent to this round-bottom flask, and the filter was washed by refluxing while heating to 88° C. in an oil bath. This washing operation was repeated three times by exchanging the ethanol, and a washed film was obtained. The washing time for each washing was 30 minutes.

The washed filter was further dried in a round-bottom flask set at 80° C. for 12 hours to form a dry film (drying process). This dry film was used as the porous metal complex-containing film according to Example 1.

The particle size distribution of the porous metal complex contained in this porous metal complex-containing film was determined by image observation using a scanning electron microscope, and the spread of the particle size distribution was in the range of 0.1 μm to 2.5 μm, with d50 being 1.0 μm. Therefore, it was found that the porous metal complex-containing film according to the present Example contains a porous metal complex with a narrow (sharp) particle size distribution of fine crystals.

Next, 30 porous metal complex-containing films according to the present Example were used as detection elements to measure gas concentration, and their detection limit was confirmed.

As described below, the product yield calculated based on the detection limit was 90%. It was thus found that the production method described in the present Example can reproducibly (i.e., with a high yield) produce a porous metal complex-containing film with high detection sensitivity when used as a detection element.

The product yield was evaluated using the measurement apparatus 100 illustrated in FIG. 1. When using the porous metal complex-containing film according to the present Example as the detection element 2 to detect trace moisture concentration in a sample gas, it was determined whether the detection element 2 could achieve a predetermined detection sensitivity (whether the detection limit was equal to or less than a predetermined value).

The conditions for this evaluation test were as follows.

An LED lamp (OSB5XNE1C1E, produced by OptoSupply) was used as the light source 3, and a photo sensor (TSL-257, produced by AMS-TAOS USA) was used as the light receiving element 4.

The sample gases used were high-purity nitrogen gas (moisture concentration<1 vppb (ppb by volume)) and a standard gas (base gas of nitrogen, moisture concentration of 10 vppm (ppm by volume); manufactured by Taiyo Nippon Sanso JFP Corp.).

In the evaluation test, high purity nitrogen gas and a gas in which a fixed amount of a standard gas was added to a high purity gas were switched at regular intervals and supplied as sample gas G to the measurement unit 1, while observing the output of the detection result of the moisture concentration.

When the standard gas was supplied, the absorbance of the porous metal complex acting as a sensor changed depending on the moisture concentration in the gas, and if a peak output corresponding to the moisture concentration could be observed, the detection limit (unit: vppb) was calculated from the ratio between the voltage intensity, which increased when switching from the high-purity nitrogen gas to the standard gas, and noise. It was then confirmed whether this detection limit was equal to or less than a certain value (in the present Example, 10 vppb) (whether the detection limit was equal to or greater than the predetermined detection sensitivity). The number of detection elements 2 (porous metal complex-containing films) having at least the predetermined detection sensitivity was determined, and based on this, the yield was calculated. The yield, determined in this way, of the porous metal complex-containing film according to the present Example was 90% as described above.

### Example 2

The present Example differs from Example 1 in that instead of using ethanol as the first polar solvent, 2-propanol (grade: special reagent grade) produced by Kanto Chemical Co., Inc. was used as the first polar solvent. Otherwise, a porous metal complex-containing film was produced and evaluated in the same manner as in Example 1.

The particle size distribution of the porous metal complex contained in the porous metal complex-containing film according to the present Example was determined by image observation using a scanning electron microscope, and the spread of the particle size distribution was in the range of 0.05 μm to 0.7 μm, with d50 being 0.4 μm. Therefore, it was found that the porous metal complex-containing film according to the present Example contains a porous metal complex with a narrow particle size distribution of fine crystals.

The product yield calculated based on the detection limit was also high at 78%.

### Example 3

The present Example differs from Example 1 in that the precipitated film in Example 1 was treated as a pre-precipitated film, which was subjected to humidification treatment (humidification process) and further to vacuum heating (second intermediate drying process). Otherwise, a porous metal complex-containing film was produced and evaluated in the same manner as in Example 1.

That is, the precursor-impregnated film produced in the same manner as in Example 1 was heated and dried in the same manner as in Example 1, and the precursor-impregnated film was then vacuum heated and dried in the same manner as in Example 1 to obtain a pre-precipitated film (the above constitutes the first intermediate drying process). Subsequently, dry argon gas was passed through a heating vessel containing ultrapure water to prepare argon gas containing water vapor at a temperature of 60° C. and a relative humidity of 40%. This argon gas was continuously supplied into a round-bottom flask containing the pre-precipitated film while creating a vacuum (reducing pressure) with a vacuum pump to humidify the pre-precipitated film (humidification process). The supply of argon gas containing water vapor was then stopped, and the round-bottom flask was heated to 100° C. again in an oil bath while being evacuated with a vacuum pump to create a vacuum inside the round-bottom flask, and vacuum-heating treatment was performed for 2 hours (second intermediate drying process) to obtain a precipitated film. After the precipitated film was obtained, a porous metal complex-containing film was produced and evaluated in the same manner as in Example 1.

The particle size distribution of the porous metal complex contained in the porous metal complex-containing film according to the present Example was determined by image observation using a scanning electron microscope, and the spread of the distribution was in the range of 0.1 μm to 0.5 μm, with d50 being 0.3 μm. Therefore, it was found that the porous metal complex-containing film according to the present Example contains a porous metal complex with a narrow particle size distribution of fine crystals.

The product yield calculated based on the detection limit was also high at 93%.

### Comparative Example 1

The present Comparative Example differed from Example 1 in that, instead of using ethanol as the first polar solvent and the third polar solvent in Example 1, dimethyl sulfoxide (grade purity: >99.5 %) produced by Fujifilm Wako Pure Chemical Corporation was used as the first polar solvent, and acetone (for high-performance liquid chromatography) produced by Kanto Chemical Co., Inc. was used as the third polar solvent, differing from Example 1. Otherwise, a porous metal complex-containing film was produced and evaluated in the same manner as in Example 1.

The particle size distribution of the porous metal complex contained in the porous metal complex-containing film according to the present Comparative Example was determined by image observation using a scanning electron microscope, and the spread of the distribution was in the range of 0.2 μm to 11 μm, with d50 being 3.5 μm. Therefore, it was found that the porous metal complex-containing film according to the present Comparative Example contains a porous metal complex with large crystals and a wide particle size distribution as compared to Examples 1 and 2.

The product yield calculated based on the detection limit was 30%, which was low compared to the above Examples.

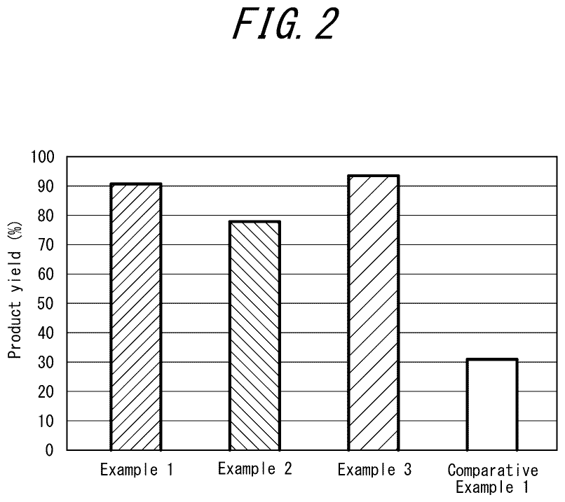

For reference in understanding the present Examples and the like, a bar graph illustrating the product yields of Examples 1 to 3 and Comparative Example 1 is illustrated in FIG. 2. As illustrated in FIG. 2, the product yields of Examples 1, 2, and 3 are significantly higher than the product yield of Comparative Example 1. In other words, according to the production methods of these Examples, it is possible to reproducibly produce high-quality porous metal complex-containing films as detection elements. This is believed to be because, compared to Comparative Example 1, the particle size distribution of the porous metal complex in these Examples could be narrowed and the particle size could be made smaller, allowing for the homogenization of the porous metal complex-containing film and thereby reducing errors between lots during production. In particular, in Example 3, the particle size distribution of the porous metal complex could be made even narrower while reducing the particle size as compared to Examples 1 and 2, which is believed to have further homogenized the porous metal complex-containing film, thereby further reducing errors between lots during production.

As illustrated in each Example above, according to the production method of the present embodiment, it is possible to produce the porous metal complex-containing film according to the present embodiment through a simple process. For example, the production of the porous metal complex-containing film does not require complex processes such as centrifugation, application of shear stress, fibrillation, or synthesizing or growing crystals using polar organic solvents and water. The porous metal complex-containing film according to the present embodiment also exhibits high sensitivity when the porous film is used as a sensor, i.e., has high detection sensitivity, and also has high response strength. Therefore, the porous metal complex-containing film according to the present embodiment is extremely useful as a detection element.

As described above, a porous metal complex-containing film that can be produced through a simple process, has a high yield during production, and is highly sensitive, and a method of producing the same, can be provided. Furthermore, a detection element using such a porous metal complex-containing film can be provided.

The configurations disclosed in the above embodiments (including other embodiments; the same applies below) can be applied in combination with the configurations disclosed in other embodiments, as long as no contradiction arises. Furthermore, the embodiments disclosed in the present specification are examples; the embodiments of the present disclosure are not limited thereto and may be modified as appropriate within a scope that does not depart from the purpose of the present disclosure.

### INDUSTRIAL APPLICABILITY

The present disclosure can be applied to a porous metal complex-containing film, a detection element provided with the same, and a method of producing a porous metal complex-containing film.

### REFERENCE SIGNS LIST

1 Measurement unit
10 Detection element holder
100 Measurement apparatus
11 Voltage measurement unit
12 Calculator
2 Detection element
4 Light receiving element
5 Light source side block
6 Light receiving side block
7 Central block
8 Flat light-transmitting member
8A Annular packing
9 Convex light-transmitting member
9A Annular packing
C Sample gas path
C1 Sample gas inflow path
C2 Sample gas outflow path
G Sample gas
L Light receiving passage
P Measurement light passage

1. A porous metal complex-containing film comprising:
a filter having a void inside and having light permeability and gas permeability; and
a porous metal complex fixed to the void, wherein
the porous metal complex contains a metal ion and an organic ligand that coordinates to the metal ion, and
a median diameter of the porous metal complex on a volume basis is 0.1 μm or more and 3 μm or less.

2. The porous metal complex-containing film according to claim 1, wherein
the metal ion is a copper ion, and
the organic ligand is benzene-1,3,5-tricarboxylic acid.

3. The porous metal complex-containing film according to claim 1, wherein the filter is cellulose fiber, glass fiber, or polytetrafluoroethylene fiber.

4. A detection element comprising the porous metal complex-containing film according to claim 1.

5. A method of producing the porous metal complex-containing film according to claim 1, the method comprising:
a solution preparation process of mixing a first polar solvent, the organic ligand, and a metal salt to prepare a precursor solution of the porous metal complex;
an impregnation process of impregnating the precursor solution into the filter to obtain a precursor-impregnated film;
an intermediate drying process of drying the precursor-impregnated film under reduced pressure to obtain a precipitated film; and
a washing process of washing the precipitated film with a second polar solvent to obtain a washed film.

6. The method of producing the porous metal complex-containing film according to claim 5, wherein the first polar solvent is methanol, ethanol, 1-propanol, 2-propanol, 1-butanol, or 2-butanol.

7. The method of producing the porous metal complex-containing film according to claim 5, wherein the metal salt is copper nitrate.

8. The method of producing the porous metal complex-containing film according to claim 5, wherein the intermediate drying process includes a humidification process of humidifying the precursor-impregnated film.

9. The method of producing the porous metal complex-containing film according to claim 8, wherein in the humidification process, the precursor-impregnated film is humidified by being exposed to a water vapor-containing gas.

10. The method of producing the porous metal complex-containing film according to claim 9, wherein a carrier gas for transporting water vapor in the water vapor-containing gas is argon, nitrogen, and air.

11. The method of producing the porous metal complex-containing film according to claim 9, wherein the water vapor-containing gas has a temperature of 40° C. or higher and 60° C. or lower and a relative humidity of 30% or higher and 70% or lower.

12. The method of producing the porous metal complex-containing film according to claim 9, wherein in the humidification process, a time for exposing the precursor-impregnated film to the water vapor-containing gas is 12 hours or more and 24 hours or less.

13. The method of producing the porous metal complex-containing film according to claim 5, wherein the intermediate drying process includes
a first intermediate drying process of drying the precursor-impregnated film to obtain a pre-precipitated film,
a humidification process of humidifying the pre-precipitated film to obtain a humidified film, and
a second intermediate drying process of drying the humidified film to obtain the precipitated film.
